(19) United States
(12) Patent Application Publication
(10) Pub. No.: US 2007/0161541 A1
(43) Pub. Date: Jul. 12, 2007

(54) Insult induced immune dissonance

(76) Inventors: Marian L. Kruzel, Houston, TX (US); Tadeusz Kruzel, Kunice (PL); Michal Zimecki, Wroclaw (PL); Istvan Boldgh, League City, TX (US)

Correspondence Address: Kurt S. Myers, 7634 Braesdale Ln., Houston, TX, 77071, US

(21) Appl. No.: 11/716,761

(22) Filed: Mar. 10, 2007

Related U.S. Application Data
(63) Continuation-in-part of application No. 11/040,963, filed on Jan. 22, 2005.
Application No. 11/040,963 is a continuation-in-part of application No. 10/140,380, filed on May 7, 2002, now abandoned.
(60) Provisional application No. 60/289,666, filed on May 9, 2001.

(51) Int. Cl. A61K 38/40

(52) U.S. Cl. 514/6

(57) ABSTRACT

The present invention is directed to the use of lactoferrin for alleviation of immune dissonance due to age-related and chronic conditions such as autoimmune disease, neurodegenerative and immune hypersensitivity conditions.

### RELATED APPLICATIONS

This application is a continuation in part of U.S. application Ser. No. 11/040,963 filed Jan. 22, 2005, entitled “Lactoferrin for age related disorders in humans”, which is a continuation in part of U.S. application Ser. No. 10/140,380, filed May 7, 2002, entitled “Lactoferrin for age related disorders in humans”, which in turn is based on provisional application No. 60/289,666 filed May 9, 2001, entitled “Method for the Use of Lactoferrin to Modulate Immune Responses in Humans and Animals”, and provisional application No. 60/782,441 filed Mar. 15, 2006, entitled “Protection against oxidative stress-induced allergic response”, each of which is incorporated herein by reference.

### FIELD OF THE INVENTION

The present invention is directed to the use of lactoferrin for alleviation of immune dissonance due to age-related and chronic conditions such as autoimmune disease, neurodegenerative and immune hypersensitivity conditions, including Alzheimer's disease, Parkinson's disease, multiple sclerosis, rheumatoid arthritis, cancer, allergy, stroke or fatigue, and its use for the manufacture of a medicament for the treatment or prevention of such disorders in humans. In particular the present invention is directed to lessening the immune dissonance in a subject having or at risk of developing such immune dissonance arising from the insult-induced oxidative stress. The present invention is based on a novel observation that lactoferrin is a useful mediator of immune responses, and in particular, effective in the slowing down of the progression or preventing the development of many chronic conditions in humans. More importantly, the present invention differentiates lactoferrin from simple iron binding mediator and provides evidence for such superior activity.

### BACKGROUND OF THE INVENTION

Lactoferrin, an iron-binding glycoprotein, is considered an important mediator in host defense against pathogenic organism. Lactoferrin is a cell-secreted mediator that bridges innate and adaptive immune function by regulating target cell response. The significance of lactoferrin in health and disease has been the subject of several reviews (Sanches L., Calvo M., Brock J H., (1992) Biological role of lactoferrin. Arch Dis Child. 67, 657-661; Lonnerdal B., Iyer S. (1995). Lactoferrin: molecular structure and biological function. Annu. Rev. Nutr. 15, 93-110). Lactoferrin has well-defined, direct antimicrobial activity (Zagulski T, Lipinski P, Zagulska A, Broniek S, Jarzabek Z. Lactoferrin can protect mice against a lethal dose of Escherichia coli in experimental infection in vivo. Br J Exp Pathol. 1989;70(6):697-704). It can also be categorized as an immunomediator during inflammatory responses. Lactoferrin is particularly active at mucosal surfaces. Because of its high concentration in human colostrum, lactoferrin has been studied extensively in host defense responses in infants (Brock J. H. Lactoferrin in human milk: its role in iron absorption and protection against enteric infection in the newborn infants. Arch. Dis. Child. 1980;55, 417-421; Howie P W., Forsyth J S., Ogston S A., Clark A., du V. Florey C. Protective effects of breast feeding. B. M. J. 1990; 300, 11-16). It is theorized that lactoferrin within human milk provides protection against pathogens during newborn adaptation to non-uterine life, and plays a role in rendering breast-fed infants more resistant to the development of microbe-induced gastroenteritis (compared to formula-fed babies). U.S. Pat. No. 4,977,137 of Nichols et al. discloses milk lactoferrin as a dietary ingredient which promotes growth of the gastrointestinal tract of human infants and newborn nonhuman animals immediately on birth. Nichols discusses the use of lactoferrin in the management of short gut syndrome, an anatomical dysfunction.

Lactoferrin has a profound modulatory action on the immune system (Zimecki M., Machnicki M., Lactoferrin inhibits the effector phase of delayed type hypersensitivity to sheep erythrocytes and inflammatory reactions to M. bovis (BCG). Arch Immunol Ther Exp 1994; 42:171-177), it promotes maturation of T cell precursors into immunocompetent helper cells and differentiation of immature B cells to become efficient antigen presenting cells (Zimecki M., Mazurier J., Spik G., Kapp J A. Human lactoferrin induces phenotypic and functional changes in splenic mouse B cells. Immunology 1995; 86:112-127). Lactoferrin is an integral part of the cytokine-induced cascade during insult-induced metabolic imbalance (Kruzel M., Harari Y., Chen Y., Castro A. G. Lactoferrin protects gut mucosal integrity during endotoxemia induced by lipopolysaccharide in mice. Inflammation 2000;24:33-44). Receptors for Lactoferrin have been identified and characterized on monocytes, B and T cells. Lactoferrin injected intravenously, intraperitoneally, or orally is quickly taken up by cells of the immune system, preferably by cells of the reticuloendothelial-system. Lactoferrin upregulates expression of leukocyte function associated-1 (LFA-1) antigen on human peripheral blood lymphocytes (Zimecki M, Miedzybrodzki R, Mazurier J, Spik G. Regulatory effects of lactoferrin and lipopolysaccharide on LFA-1 expression on human peripheral blood mononuclear cells. Arch Immunol Ther Exp 1999;47:257-264). As presented in FIG. 1, lactoferrin can modulate the outcomes of acute inflammation, which is fundamentally a protective response to cell injury as disclosed in PCT application number WO 98/50076, entitled “Methods for Preventing and Treating the Insult-Induced Metabolic imbalance in humans and other Animals”, filed May 3, 1997, all of which is incorporated herein by reference.

The role of lactoferrin in modulating both the acute and chronic inflammation is under active investigation. By virtue of high affinity to iron, lactoferrin is considered an important component of nonspecific host defense system against various pathogens in humans. A high level of lactoferrin in plasma has been suggested to be a predictive indicator of sepsis-related morbidity and mortality (Bayens R D., Bezwoda W R. Lactoferrin and the inflammatory response In: Lactoferrin: Structure and Function, eds. T. W. Hutchens et al., Plenum Press, 1994; pp. 133-141). In addition, progression in chronic inflammatory disorders, such as Alzheimer's disease, or autoimmune disorder, such as multiple sclerosis, seems not to be interrupted by lactoferrin elevation in various physiological fluids. Although, the endogenous production of lactoferrin is increased in these disorders, it is either not sufficient, or does not trigger the pathway(s) of molecular events to aid a defense system against the disorder. Thus, the use of exogenous lactoferrin for treatment of such conditions would not be obvious. It is possible though that the exogenous lactoferrin, especially when given orally, transduces different signaling pathways than the endogenous lactoferrin molecule.

Under normal physiological conditions, the rate and magnitude of reactive oxidants formation is balanced by the rate of their elimination. An imbalance between reactive oxidants production and antioxidant defense results in oxidative stress, which may lead to the oxidative cell injury (Touyz R M. “Oxidative stress and vascular damage in hypertension”. Curr Hypertens Rep. 2000;2(1):98-105). Oxidative stress can contribute to many diseases including fatigue, sepsis, autoimmune diseases, hypersensitivity, cancer, neurodegenerative diseases, heart attack and stroke. Transitional metals have been considered as key factors in the oxidative stress. In particular, traces of iron can be detrimental to physiological processes under reactive oxygen conditions. Iron is in a center of the reactive oxygen species control. It has the ability to catalyze two step process known as the Haber-Weiss reaction (FIG. 2). In the first reaction a superoxide molecule reacts with iron (3⁺) salt to form iron (2⁺) salt and ground state oxygen. The second reaction is known as the Fenton reaction. In this reaction iron (2⁺) salt reacts with hydrogen peroxide to form iron (3⁺) salt, the hydroxyl radical and alcohol.

In normal physiological conditions the production and neutralization of these reactive oxygen species (ROS) depend on the efficiency of key enzymes, including superoxide dismutase (SOD), catalase (CAT) and glutathione peroxidase (GPX). If the process of neutralization of ROS is not efficient, it can contribute to development of oxidative stress (e.g. lipid peroxidation). Although, endogenous lactoferrin participates in these processes at cellular level it is not understood how exogenous lactoferrin would contribute to these molecular events (FIG. 2).

Reactive oxygen species are capable of catalyzing morphological changes to proteins, in both beneficial and non-beneficial ways. The ability of a cell to control these changes in oxidation and resulting protein effects is very important for species survival. Recently, intermediates in the lipid peroxidation process have shown the ability to inactivate and modify proteins. Lipid peroxidation is tentatively defined as the oxidative deterioration of polyunsaturated lipids. These fatty acids provide mobility and fluidity to the plasma membrane, properties which are known to be essential for the proper function of biological membranes. The process of lipid peroxidation is a step-wise process with an initiation and subsequent propagation reactions. Iron and other transitional metals help to initiate the process by forming alkeoxy or peroxy radicals upon reaction with oxygen species. The fatty acids are reduced to reactive aldehydes and hydrocarbons. In general, the damaging consequences of lipid peroxidation are expressed as a decrease in the fluidity of the membrane and subsequent increase in its permeability to substances which normally do not pass.

The nervous system, including the brain, spinal cord, and peripheral nerves, is rich in both unsaturated fats and iron (Halliwell. Reactive oxygen species and the central nervous system. J Neurochem. 1992;59(5):1609-23). The high lipid content of nervous tissue, coupled with its high metabolic activity, makes it particularly susceptible to oxidant damage. The high level of brain iron may be essential to oxidative stress via the iron-catalyzed formation of reactive oxygen species.

In the age-related and chronic disorders that develop over decades, many chemical species as well as pathophysiological conditions are involved. The major threat comes from the oxidative stress. The generation of the reactive oxygen species can lead to immediate damage or death of cells in various tissues (Gutteridge. Hydroxyl radicals, iron, oxidative stress, and neurodegeneration. Ann N Y Acad Sci. 1994;738:201-13). There is substantial evidence that oxidative stress is a causative factor in the pathogenesis of major neurodegenerative diseases, including Parkinson's disease (Ebadi M, Srinivasan S K, Baxi M D. Oxidative stress and, antioxidant therapy in Parkinson's disease. Prog Neurobiol. 1996;48(1):1-19), Alzheimer's disease (Markesbery W R, Carney J M. Oxidative alterations in Alzheimer's disease. Brain Pathol. 1999;9(1):133-46.; Behl Vitamin E and other antioxidants in neuroprotection. Int J Vitam Nutr Res. 1999;69(3):213-9), and amyotrophic lateral sclerosis (Olanow and Arendash Metals and free radicals in neurodegeneration. Curr Opin Neurol. 1994;7(6):548-58.; Simonian and Coyle Oxidative stress in neurodegenerative diseases. Annu Rev Pharmacol Toxicol. 1996;36:83-106) as well as in cases of stroke, trauma, and seizures (Coyle and Puttfarcken. Oxidative stress, glutamate, and neurodegenerative disorders. Science. 1993;262(5134):689-95.; Facchinetti F, Dawson V L, Dawson T M. Free radicals as mediators of neuronal injury. Cell Mol Neurobiol. 1998;18(6):667-82) or rheumatoid arthritis, fatigue and cancer (Kovacic P, Jacintho J D. Mechanisms of carcinogenesis: focus on oxidative stress and electron transfer. Curr Med Chem 2001;8(7):773-96).

Also, there is ample evidence that allergic disorders, such as asthma, rhinitis, and atopic dermatitis, are mediated by oxidative stress (Bowler R P., Capro J D. (2002): Oxidative stress in allergic respiratory diseases J Allergy Clin Immunol. 110:349-56). In fact, the oxidative stress-induced immune hypersensitivity indicates a shift in immunostasis towards the Th2 responses. The Th1/Th2 balance is responsible for coordinating the immune system and become very important during aging processes, including the development of autoimmune, neurodegenerative and immune hypersensitivity disorders.

The physiological function of lactoferrin is still under investigation. For example, in a review by Roy D. Byens and Wemer R. Bezwoda entitled “Lactoferrin and the inflammatory response” and published in the book: Lactoferrin: Structure and Function, pp 133-141, (1994), a relationship between plasma lactoferrin and granulocyte activity in sepsis is discussed, yet not completely understood.

Similarly, marked elevation of lactoferrin has been noted in the cerebrospinal fluid of patients with acute cerebrovascular lesions and other pathological lesions in variety of neurodegenerative disorders (Penco S, Villaggio B, Mancardi G, Abbruzzese M, Garre C. A study of lactoferrin and antibodies against lactoferrin in neurological diseases. Adv Exp Med Biol. 1998;443:301-40). Based on this observation the use of exogenous lactoferrin in patients who overexpress its own lactoferrin would not be scientifically justified.

In another review entitled “The role of lactoferrin as an anti-inflammatory molecule” by Bradley E. Britigan, Jonathan S. Serody, and Myron S. Cohen and published in the book: Lactoferrin: Structure and Function, pp 143-156, (1994), the role of lactoferrin in inflammation is suggested to be played at two different levels: (i) as an antioxidant, capable of binding free iron, and (ii) as an endotoxin scavenger, capable of reducing lipopolysaccharide (LPS)-induced toxicity.

In yet another article entitled “Lactoferrin in infant formulas: effect on oxidation”, by Satue-Gracia M T, Frankel E N, Rangavajhyala N, German J B., and published in J Agric Food Chem. 2000;48(10):4984-90, authors emphasize the ability of lactoferrin to control oxidation via iron-sequestration. However no evidence is presented for iron independent activity of lactoferrin in vivo in relation to oxidation and particularly oxidative stress.

Relevant patents are also silent as to the use of lactoferrin for prevention or therapy of autoimmune or neurodegenerative disorders in humans and animals. U.S. Pat. No. 5,240,909 of Nitsche relates to the use of lactoferrin as an agent for the prophylactic and therapeutic treatment of the toxic effects of endotoxins. Nitsche discloses that the lactoferrin used according to his invention has the ability to neutralize endotoxin and must have bound to it either iron or another metal to be effective. U.S. Pat. No. 5,066,491 of Stoft et al. encompasses a method of disease treatment utilizing a therapeutically effective product produced from ordinary milk whey.

Despite large number of studies on lactoferrin, there is no disclosure that it can function as an insuft-induced immune dissonance mediator to reduce the debilitating conditions in the autoimmune, neurodegenerative and immune hypersensitivity disorders such as Alzheimer's, Parkinson's, multiple sclerosis, rheumatoid arthritis, cancer, allergy, stroke or fatigue. The knowledge about endogenous lactoferrin is not supporting the clinical effects of exogenous lactoferrin as found in the present invention. For example, autoantibodies to lactoferrin are commonly found in many autoimmune disorders, including multiple sclerosis (Penco S, Villaggio B, Mancardi. G, Abbruzzese M, Garre C. A study of lactoferrin and antibodies against lactoferrin in neurological diseases. Adv Exp Med Biol. 1998;443:301-40) and rheumatoid arthritis (Locht H, Skogh T, Kihlstrom E. Anti-lactoferrin antibodies and other types of anti-neutrophil cytoplasmic antibodies (ANCA) in reactive arthritis and ankylosing spondylitis. Clin Exp Immunol. 1999;117(3):568-73). In fact, the presence of these antibodies has been suggested to be used as marker for the inflammatory disorders. Based on this observation the use of exogenous lactoferrin in patients with autoantibodies to lactoferrin would not be scientifically justified.

Here we demonstrate a novel approach for use of lactoferrin to modulate insult induced immune dissonance in age-related and chronic disorders, including neurodegenerative, autoimmune and immune hypersensitivity conditions, via both iron-dependent and iron-independent oxidative stress mechanism. More importantly, the present invention differentiates lactoferrin from simple iron binding mediator as evidenced by the examples herein.

### SUMMARY OF THE INVENTION

The method of the present invention provides a novel use of lactoferrin to modulate the molecular events during development of age-related and chronic disorders including autoimmune, neurodegenerative and immune hypersensitivity disorders in humans. More specifically, the present invention is directed to the use of lactoferrin for alleviation of insult induced immune dissonance in subjects having or being at risk of such immune dissonance as exemplified by Alzheimer's disease, multiple sclerosis, rheumatoid arthritis, allergy, stroke or chronic fatigue syndrome, and lactoferrin use for the manufacture of a medicament for the treatment or prevention of such disorders.

### BRIEF DESCRIPTION OF THE DRAWINGS

FIG. 1 Illustrates molecular events during development of acute inflammation.

FIG. 2 Illustrates cellular mechanisms of iron-dependent ROS generation.

FIG. 3 Illustrates LF effect on apoptosis. Cells were pre-treated with lactoferrin and GO (300 ng per ml) was added. Caspase-3 activities were determined in clarified cell lysates using colorimetric assays (R&D Systems, Inc). In this assay, changes in O.D. at 405 nm are proportional with activity of caspase-3. Each data point represents the mean of three independent experiments.

FIG. 4 Illustrates LF effect on RWE-mediated increase in intracellular ROS levels in cultured cells and BAL fluid. A, A549 cells were loaded with H₂DCF-DA for 15 min and challenged with RWE plus NAD(P)H. Changes in intracellular DCF fluorescence were determined fluorimetrically. G-ox, which generates O₂⁻, was used as control. B, Effect of LF on H₂O₂ levels excreted into the medium of RWE-treated A549 cells determined by Amplex® Red assays. C, Changes in ROS level in normal human bronchial epithelial cells. LF decreases H₂O₂ (D) and 4-HNE+MDA (E) levels in the BAL of RWE-challenged mice (n=5-8). Each data point represents the mean from three or more independent experiments, ±SEM. **P<0.01, ***P<0.001; ****P=0.0001.

FIG. 5 Illustrates LF effect on RWE-induced allergic airway inflammation. Accumulation of eosinophils (A) and total inflammatory cells (B) in BAL fluids was assessed at 72 hr after RWE challenge (n=6-8 mice per group). Results are means±SEM. *P=0.05; ***P=0.001; ****P<0.0001.

FIG. 6 Illustrates LF effect on the RWE-induced accumulation of inflammatory cells into subepithelium and goblet cell formation. A, Microscopic visualization of eosinophilic infiltration and goblet cell metaplasia. The mice were sacrificed 72 hr after RWE challenge, and their lungs were processed and sections were stained with hematoxylin and eosin or PAS. Upper panels: inflammatory cell infiltration in peribronchial and perivascular regions. Lower panels: goblet cells metaplasia. Images are representative of serial sections from the lungs of seven mice in each group. B, Morphometric quantification of peribronchial inflammatory cell infiltration. C, Quantification of goblet cell metaplasia.

FIG. 7 Illustrates LF effect when added at the time of allergen challenge. Accumulation of eosinophils in BAL fluids was assessed at 72 hr after challenge (n=6-8 mice per group). Results are means±SEM. **P<0.01; ***P=0.001; ***P=0.0001.

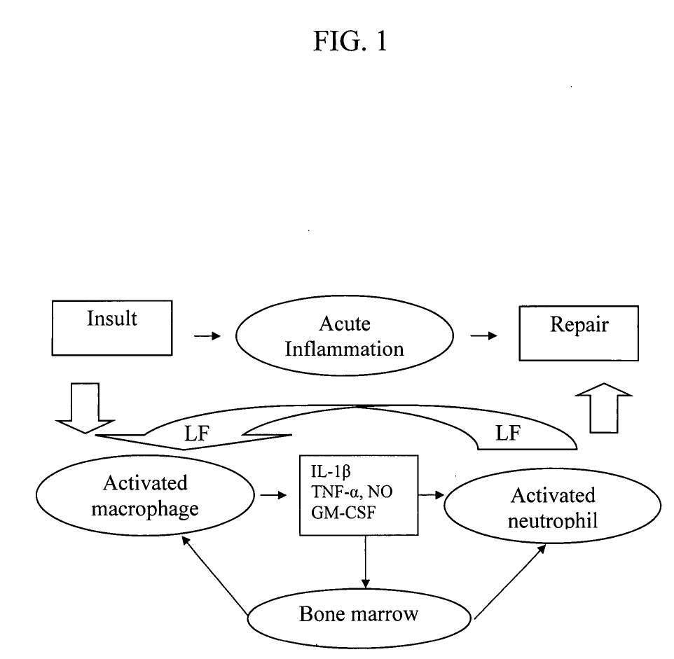

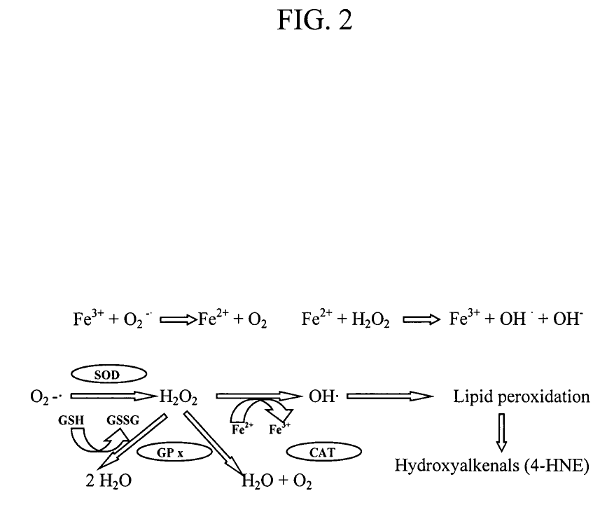

Table 1. Illustrates clinical data relevant to lactoferrin treated Alzheimer's patients.

### DESCRIPTION OF THE PREFERRED EMBODIMENTS

According to the present invention exogenous lactoferrin is used to modulate the molecular events during development of the age-related and chronic disorders, including autoimmune, neurodegenerative and immune hypersensitivity disorders in humans. In particular, lactoferrin is used to alleviate immune dissonance by assisting in the development of T-helper cell polarization during the insult induced oxidative stress in humans.

Also, according to the present invention exogenous lactoferrin is used to modulate the Th1/Th2 balance in the context of immune homeostasis. Although, many pathological phenomena have been correlated with ROS, the role of oxidative stress in such chronic disorder-related decline or increase of T-cell activity is not yet clear. Still, according to the present invention lactoferrin is used to counterbalance allergen-reactive Th2 responses, also known as type 1 hypersensitivity (immediate) including allergy. It is a major pleiotropic mediator that directly assists in the development of T-helper cells polarization.

The present invention is based on the observation from both animal and human clinical results obtained from patients with various disorders. In all examples lactoferrin was found effective, specifically including the prevention and slowing down of the progression of the disease. According to the present invention lactoferrin is used to restore and maintain immune homeostasis, in particular, related to the central nervous system health and disease. The present invention has broad implications in the alleviation, treatment, or prevention of many age-related disorders including chronic autoimmune, neurodegenerative or immune hypersensitivity (allergy) disorders, which are exemplified hereto:

Allergy. Allergy is defined as a hypersensitivity of the body's immune system in response to exposure to antigens, such as foods, pollen, dust, or certain drugs. A severe form of allergy is called anaphylactic shock, which is considered as a medical emergency. Symptoms of allergy are various and may include skin rashes, swelling, and difficulties to breathing. Symptoms of anaphylactic shock may include dizziness, loss of consciousness, swelling of the tongue and breathing tubes, blueness of the skin, low blood pressure, and death.

Multiple sclerosis (MS). MS is a disease of the central nervous system identifiably by progressive symptoms, and pathologically by scattered areas of demyelination affecting the brain, spinal cord and optic nerves. Generally, individuals note the first signs between the ages of 15 and 50. Affected patients encounter bouts of inflammatory demyelination producing the classic course of the disease of exacerbation—remittance.

Lupus. Lupus is a chronic inflammatory disease of uncertain origin, affecting many systems of the body, characterized by a rash on the face and other areas exposed to sunlight, involving the vascular and connective tissues of many organs, and accompanied by serologic abnormalities. Lupus is a chronic (long-lasting) autoimmune disease where the immune system, for unknown reasons, becomes hyperactive and attacks normal tissue.

Amyotrophic lateral sclerosis (ALS). ALS, also known as Lou Gehrig's disease, is a progressive disease of the nervous system. ALS attacks motor neurons, which are among the largest of all nerve cells in the brain and spinal cord. These cells send messages to muscles throughout the body. In ALS, motor neurons die and the muscles do not receive these messages. As a result, muscles weaken as they lose their ability to move. Eventually, most muscle action is affected, including those which control swallowing and breathing, as well as major muscles in the arms, legs, back and neck. There is, however, no loss of sensory nerves, so people with ALS retain their sense of feeling, sight, hearing, smell and taste. According to the National Institutes of Health, some 4,600 people in the United States are newly diagnosed with ALS each year.

Chronic Fatigue Syndrome (CFS). CFS is a condition of prolonged and severe tiredness or fatigue that is not relieved by rest and is not directly caused by other conditions. The exact cause of chronic fatigue syndrome is unknown. Some researchers suspect it may be caused by a virus, such as human herpes virus-6 (HHV-6). However, no distinct viral cause has been identified. Recent studies have shown that chronic fatigue syndrome may be caused by nonspecific inflammation in the nervous system; and that this may trigger some sort of autoimmune process. Other factors such as age, prior illness, stress, environment, or genetic disposition may also play a role. Symptoms of CFS are similar to those of most common viral infections (muscle aches, headache, and fatigue), often developing within a few hours or days and lasting for several months or more. Although common fatigue is different from CFS, both are oxidative stress-driven disorders.

Rheumatoid arthritis (RA). RA is a systemic autoimmune disease which initially attacks the synovium, a connective tissue membrane that lines the cavity between joints and secretes a lubricating fluid. The cause of rheumatoid arthritis is unknown. In fact, it is possible that there is no single cause of RA. Infectious, genetic, and hormonal factors may play a role. The disease can occur at any age, but the peak incidence of disease onset is between the ages of 25 and 55. The incidence increases with age. The onset of the disease is usually gradual, with fatigue, morning stiffness lasting more than one hour, diffuse muscular aches, loss of appetite, and weakness. Eventually, joint pain appears, with warmth, swelling, tenderness, and stiffness of the joint after inactivity.

Alzheimer's Disease (AD). AD is a neurodegenerative disorder mainly characterized by the progressive and irreversible loss of nerve cells (neurons) located in a specific brain area, the hippocampus. AD is a disease that attacks the brain and results in impaired memory, thinking and behavior. The destruction of nerve cells leads to a decrease in neurotransmitters. The correct balance of neurotransmitters is critical to the brain. Three neurotransmitters commonly affected by AD are acetylcholine, serotonin, and norepinephrine. Memory impairment is a necessary feature for the diagnosis. Change in one of the following areas must also be present: language, decision-making ability, judgment, attention, and other related areas of cognitive function and personality. Alzheimer's disease (AD) is a slowly progressive form of dementia.

Parkinson's Disease (PD). PD is a degenerative disease that often manifests itself late in life and is marked by abrupt motions, muscle tremors and a peculiar gait. People who suffer from this disease, once thought to be strictly neuromuscular, lose neurons from a part of the brain called the substantia nigra that produces the neurotransmitter dopamine, which helps brain cells communicate with one another. Parkinson's patients also experience a slowing of some cognitive functions and have difficulty with complex tasks.

Hantington's Disease (HD). HD is a genetic disease involving the degeneration of nervous system cells, including brain cells, beginning at around age 30. HD is characterized initially by bradykinesia and rigidity then choreiform movements.

Creutzfeldt-Jakob Disease (CJD). CJD, human transmissible spongiform encephalopathies have been transmitted to primates and to other animals through cell-free injections of infected brain tissue. Spongiform encephalopathies occur in several mammalian species. Scrapie affects sheep, and bovine spongiform encephalopathy or mad cow disease occurs primarily in cows. Kuru, which affects humans, is associated with cannibalism in New Guinea natives. C-J syndrome and Gerstmann-Straussler-Schenker syndrome, which affect humans, appear to occur through both genetic and infectious routes, as known for scrapie. The infectious agent has been characterized and is resistant to inactivation by ultraviolet radiation, formalin, heat and enzymes which denature nucleic acids. It can be inactivated (i.e. its infectivity destroyed) by proteases and other treatments that denature proteins.

Stroke. Stroke is a cardiovascular disease that affects the blood vessels supplying blood to the brain. It is also sometimes called brain attack. A stroke occurs when a blood vessel bringing oxygen and nutrients to the brain bursts or is clogged by a blood clot or some other particle. Deprived of oxygen, nerve cells in the affected area of the brain can't function and die within minutes. And when nerve cells can't function, the part of the body controlled by these cells can't function either. There are four main types of stroke: two caused by blood clots or other particles, and two by hemorrhage. Cerebral thrombosis and cerebral embolism are by far the most common, accounting for about 70-80 percent of all strokes. They're caused by clots or particles that plug an artery. Cerebral and subarachnoid hemorrhages are caused by ruptured blood vessels. They have a much higher fatality rate than strokes caused by clots.

Cancer. Cancer is defined as an uncontrolled growth of abnormal cells which have mutated from normal tissues. Cancer can kill when these cells prevent normal function of affected vital organs or spread throughout the body to damage other key systems. There are at least 200 different kinds of cancers, which can develop in almost any organ. Typically, the growth of cells in the body is strictly controlled—new cells are made as needed to replace older ones or to perform needed functions. If the balance of cell growth and death is disturbed, cancer may occur. Problems in the regulation of cell growth can be caused by abnormalities of the immune system, which normally would detect and stop aberrant growth. Other potential causes of cancer include radiation, sunlight, tobacco, certain viruses, benzene, certain poisonous mushrooms, and aflatoxins amongst many others.

Lactoferrin for use in a present invention may be human lactoferrin from human breast milk or extracted from milk of other animals such as bovine lactoferrin from cow's milk or whey. Due to severe limitations on availability of large quantities of human breast milk and the FDA requirements, it may be difficult to develop a commercial production of clinically acceptable natural human lactoferrin. Consequently, recombinant DNA technology is considered the best solution to obtaining large quantities of reliable human or bovine lactoferrins which would be consistent in production, uniform in its biological properties, and non-pathogenic. Of particular interest for systemic human applications would be a human lactoferrin produced in an expression system providing human type glycosylation, such as described by Choi B K, Bobrowicz P, Davidson R C, Hamilton S R, Kung D H, Li H, Miele R G, Nett J H, Wildt S, Gerngross T U. Use of combinatorial genetic libraries to humanize N-linked glycosylation in the yeast Pichia pastoris. Proc Natl Acad Sci USA. 2003;100:5022-5027.

The preferred recombinant lactoferrin is lactoferrin expressed in a yeast expression system such as Pichia pastoris or Hansenula polymorpha, or in a eukaryotic expression system. The preferred lactoferrin is described in U.S. Pat. No. 6,066,469, entitled “Cloning, Expression and Uses of Human Lactoferrin” and its two divisional applications U.S. Pat. No. 4,277,817 B1 and 6,455,687 B1, both entitled “Human lactoferrin”. Other recombinant lactoferrins are described in U.S. Pat. Nos. 5,571,691; 5,571,697; and 5,571,896, all of which are incorporated herein by reference.

A preferred bovine lactoferrin is lactoferrin derived from cow's milk, which may be obtained as partially iron saturated form (typically less than 20% metal loading) from commercial sources, including DMV International Nutritionals, Frasier, N.Y.; Glanbia Foods, Inc., Richfield, Id.; Tatua Nutritionals, New Zealand: or Morinaga Milk Industry Co., Ltd., Japan. The characteristics of such preferred lactoferrin is presented in Example 1, only for the purpose of illustration.

Lactoferrin is administered in accordance with the present invention either systemically (intravenously, intramuscularly) or orally, in the form of a powder, solution or gel, as an aid to treat or prevent metabolic imbalance. Preferable formulations or medicaments of the present invention comprise lactoferrin alone or in combination with pharmaceutical or nutritional carriers such as, water, saline, starch, maltodextrin, pullulan, silica, talcum, stearic acid, its magnesium or calcium salt, polyethyleneglycol, arabic, xanthan or locoust bean gums and fatty emulsions and suspensions that will be readily apparent to the skilled artisan. The lactoferrin is preferably present in the formulation at a level of 0.01 milligram to 2 milligram, more preferably between 0.1 to 1 milligram, based on 1 milliliter or 1 gram of the carrier. An effective amount of lactoferrin varies depending on the individual treated, severity of the metabolic imbalance and the form of administration. Preferable in treating individual, a single or twice daily dose of 0.01 milligram to 20 milligrams, more preferable 0.1 milligram to 1 milligram of lactoferrin per kilogram of body weight is administrated. Lactoferrin can also be delivered as a liposomal formulation, including transdermal patches.

According to the present invention, lactoferrin can be incorporated in formulation with any drug adjuvant therapy and delivered alone or simultaneously per os, intravenously, intraperitonealy, intraarterialy, intramascularly, subcutanoeusly, transdermally, or as an intranasal spray, or intrabroncheal inhalation mist, at the effective concentration ranges set forth herein above. Preferred formulations or medicaments of the present invention comprise incorporating the lactoferrin into chewable tablets as illustrated in Example 2 or liquid formula as illustrated in Example 3 and 4.

### EXAMPLE 1—Bovine Milk Lactoferrin (BLF)

Bovine milk lactoferrin is isolated and purified from cow's milk (at least 80% pure as per polyacrylamide gel electrophoresis) with less than 20% iron saturation. BLF is free of Coliform bacteria, Salmonella and pathogenic Staphylococcus and not toxic for animals when orally administered at 2 g/kg/day for several weeks.

### EXAMPLE 2—Lactoferrin Chewable Tablets

Tablets are made from the following powdered ingredients, mixed in a commercial mixer: 95.45 parts dextrose; 2.97 parts BLF; 0.6 part citric acid; 0.34 part orange flavor; 0.07 part orange color, and mixed for 10 minutes. Then, 0.53 part of calcium stearate is added for additional 5 minutes of mixing. Each of the procedures should be performed with precautions against exposure to the powders and dusts that are formed, and particularly against their inhalation. The tablets (25 mg of BLF per tablet) are formed by direct compression with 4,000 pounds to obtain hardness of ˜180 Newtons, a characteristic of chewable tablets.

### EXAMPLE 3—Lactoferrin Liquid Formula for the Enteral Applications

The liquid form of lactoferrin is made from the following powdered ingredients, mixed with water in a commercial mixer: 92.00 parts water; 5.00 parts dextrose; 2.97 parts BLF; 0.03 part orange flavor, and mixed for 10 minutes. Each of the procedures should be performed with precautions against exposure to the powders and dusts that are formed, and particularly against their inhalation. The solution, which is an equivalent of 2.97% lactoferrin in 5% dextrose is packed into a sterile plastic bags, disposable pouches, spray containers or bottles, all under the sterile conditions. Lactoferrin as per Example 3 is given to subjects by mouth, through feeding tube or by oral, nasal or alveolar spray.

### EXAMPLE 4—Lactoferrin Liquid Formula for the Parenteral Applications

The liquid form of lactoferrin is made from the following powdered ingredients, mixed with water in a commercial mixer: 92.00 parts water; 5.00 parts dextrose; 3.00 parts lactoferrin and mixed for 10 minutes. Each of the procedures should be performed with precautions against exposure to the powders and dusts that are formed, and particularly against their inhalation. The caloric value of such solution is less than 200 kcal/L and the osmolarity is less than 300 mOsmol/L (calc.). The solution pH is between 4.3 and 6.8. The solution, which is an equivalent of 3% lactoferrin in 5% dextrose is filter sterilized using 0.22 μn filter, such as Nalgene product, than it is packed into a sterile plastic bags, disposable pouches, or vials, all under the sterile conditions. Lactoferrin as per Example 4 is given to subjects parenterally for systemic administration using intravenous, intramascular or subcutaneous injections.

### EXAMPLE 5—Lactoferrin Reduces Apoptosis

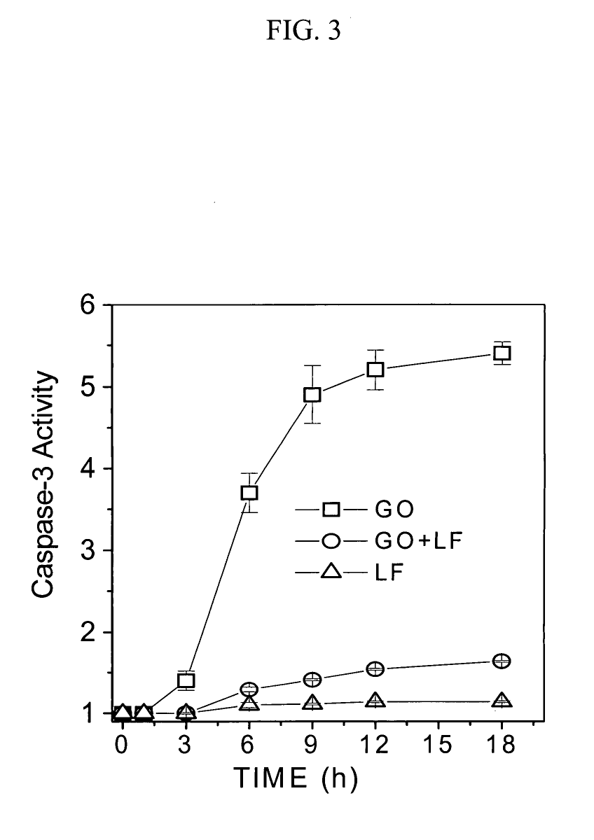

Apoptosis can be measured in U937 cells maintained in RPMI1640 (GIBCO-Invitrogen, Inc.) medium. The growth medium is supplemented with 10% FBS (Sigma-Aldrich Inc), glutamine (292 mg/L), penicillin (100 U/ml) and streptomycin (100 μg/ml). Cells are pre-treated with lactoferrin (125 or 250 μg/ml) or N-acetyl-L-cysteine (as control; 10 mM) for 3 h at 37° C. in a humified 5% CO2 atmosphere. Pretreated cells are exposed to glucose oxidase (GO) (500 ng per ml: this concentration killed cells via apoptosis, determined in preliminary studies) and activation of caspase 3 is determined colorimetrically. Briefly, cells (0, 1, 3, 6, 9, 12, and 18 h post-treatment with GO (500 ng per ml), are collected by centrifugation (1,000 rpm, at 4° C., for 10 min). The pellets are lysed in ice-cold lysis buffer and clarified by centrifugation (14,000 rpm, at 4° C., for 15 min). Enzymatic reactions are carried out in 96-well plates after addition of cell supernatant, reaction buffer and appropriate caspase substrate. Caspase activity is determined by measuring the change in absorbance at 405 nm (FIG. 3). Accordingly, lactoferrin reduced apoptosis by 80%.

To further characterize apoptosis, flow cytometric analysis was performed on cells treated with lactoferrin, GO and their combination after AnnexinV-FITC staining. Using a FACScan flow cytometer. Again, lactoferrin reduced apoptosis by 80%.

### EXAMPLE 6—Lactoferrin Reduces Immune Hypersensitivity

Cell cultures: A549 bronchial epithelial cells were purchased from American Type Culture Collection—ATCC, (Rockville, Md., USA). The A549 cells were cultured in F-12 Kaighn's-modified medium. Primary normal human bronchial epithelial (NHBE) cells obtained from Cambrex Bio Science (Walkersville, Md., USA) were cultured in BEGM® BulletKit® medium supplied by the manufacturer. The culture media were supplemented with 10% heat-inactivated fetal bovine serum (FBS), L-glutamine (2 mM), penicillin (100 U/ml), and streptomycin (100 μg/ml).

Animals: BALB/c mice were purchased from Harlan Sprague-Dawley (San Diego, Calif., USA). All animal experiments were performed according to the National Institutes of Health Guide for Care and Use of Experimental Animals.

Sensitization and challenge of animals: Eight-week-old female animals were sensitized with RWE as previously described. Briefly, mice were sensitized with two intraperitoneal administrations of endotoxin-free RWE (Greer Laboratories, Lenoir, N.C., USA), 150 μg/100 μl/injection, combined in a 3:1 ratio with Alum adjuvant (Pierce Laboratories, Rockford, Ill., USA) on days 0 and 4. On day 11, parallel groups of mice (n=6-8) were challenged intranasally with RWE (100 μg), iron-free lactoferrin (LF; 100 μg), iron-saturated lactoferrin LF^{Fe} (100 μg), RWE (100 μg)+LF (100 μg), or RWE (100 μg)+LF^{Fe} (100 μg). Some properties of LF are similar to DFO. For example, DFO binds iron 1:1 stoichiometrically, has impact on cell cycle, DNA synthesis, regulates gene expression and possess anti-proliferative as well as anti-inflammatory effects and has been utilized in clinical practice. Therefore, parallel groups of mice were challenged intranasally with RWE (100 μg), RWE (100 μg) plus DFO (100 μg) or DFO (100 μg) alone. Mice were also challenged with Amb a 1 alone (25 μg), G-ox (50 μU) alone or G-ox+Amb a 1. Control groups of mice were challenged with equivalent volumes of PBS.

Evaluation of allergic inflammation: To evaluate inflammation, animals from all experimental groups were euthanized on day 14 with ketamine (135 mg/kg body wt) and xylazine (15 mg/kg body wt), and the lungs were lavaged with two 0.8-ml aliquots of ice-cold PBS. The cells were collected by centrifugation (1000 g, for 10 min at 4° C.) and resuspended in one ml of PBS, and total cell counts were determined. Differential cell counts were performed on cytocentrifuge preparations stained with hematoxylin and eosin. After bronchoalveolar lavage (BAL), the lungs were fixed with 4% paraformaldehyde, embedded in paraffin, and sectioned to 5 μm. Lung sections were stained with hematoxylin and eosin. Perivascular and peribronchial inflammation and cell composition in the BAL were evaluated by a pathologist blinded to treatment groups to obtain data for each lung. To objectively quantify cellular (eosinophilic) infiltrations of lung sections morphometric analyses were done using NIKON Eclipse TE 200 UV microscope operated via Metamorph™ software (Version 5.09r, Universal Imaging, Downingtown, Pa., USA). Images were obtained from four different levels per lung (three animals per group) and reassembled using the montage stage stitching algorithm of the Metamorph™ software. The integrated morphometric analysis function was used to transform total pixel area of the field light intensity from the sections into μm² units after calibration. Mucin production in the epithelial cells was assessed by periodic acid Schiff (PAS)-staining of formalin-fixed, paraffin-embedded lung sections. The stained sections were analyzed as above and representative fields were photographed with a Photometrix CoolSNAP Fx camera mounted on a NIKON Eclipse TE 200 UV microscope.

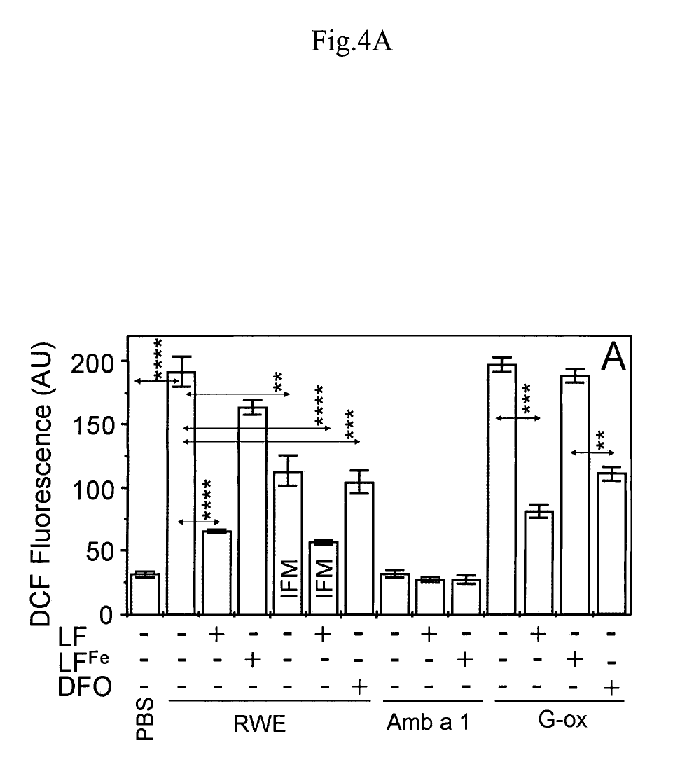

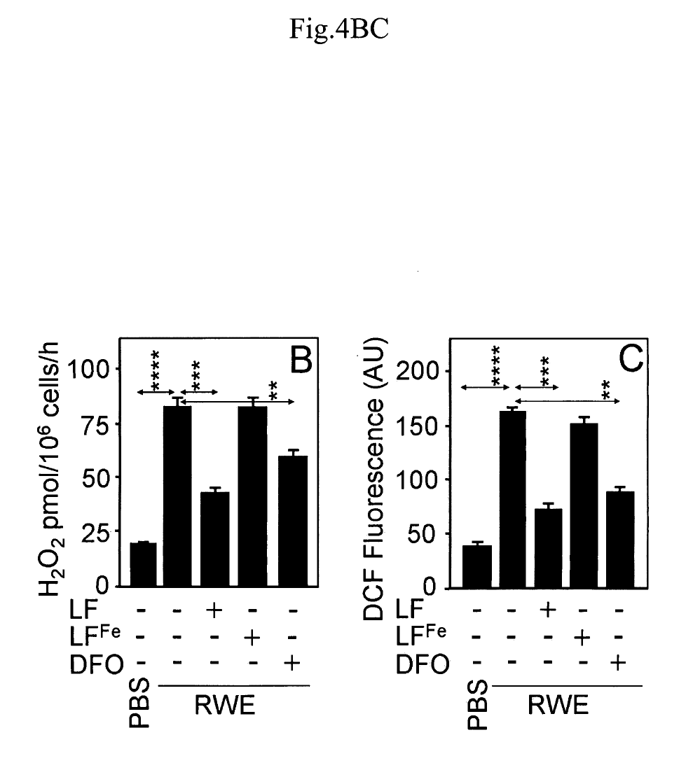

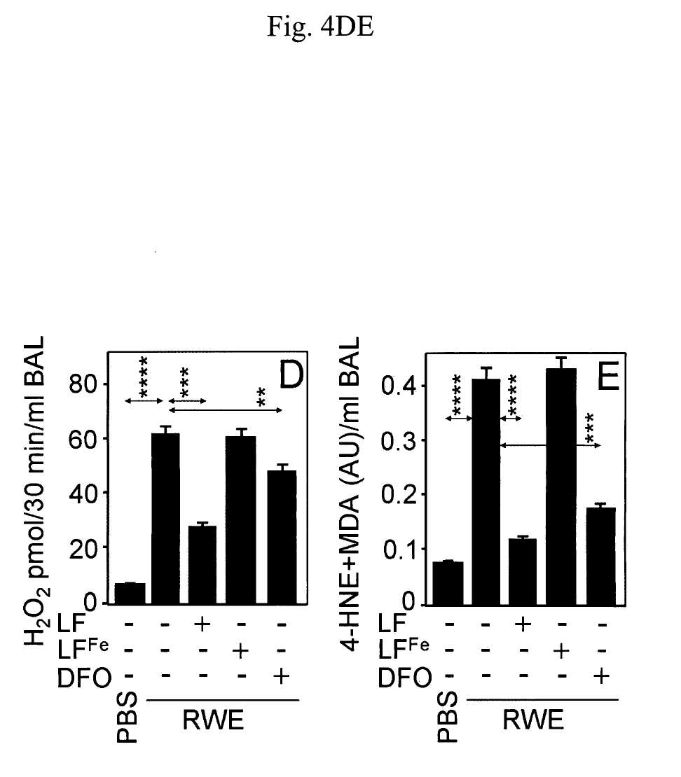

Effect of lactoferrin on RWE-mediated increase in intracellular ROS level in cultured cells: We have recently demonstrated that NAD(P)H oxidases intrinsic to pollen grains generate O₂⁻, which serves the basis of a rapid increase in oxidative stress levels in cultured bronchial epithelial (A549) and lining cells of airway and conjunctival epithelium. In this study, first we examined the effect of LF on RWE-mediated changes in intracellular ROS levels. A549 cells were grown in iron-containing medium and LF or LF^{Fe} was added for 30 min followed by loading cells with H₂DCF-DA. Cells were then exposed to 125 μg/ml RWE plus 100 μM NAD(P)H, and changes in DCF fluorescence were determined. LF (100 μg/ml), but not LF^{Fe} (100 μg/ml), significantly (P<0.001) decreased formation of fluorescent DCF (FIG. 4A). Optimal concentration of LF was determined in preliminary studies (data not shown). In control experiments G-ox (50 μU/ml), which primarily produces O₂⁻, induced a comparable increase in ROS levels. When A549 cells were RWE-treated in iron-free medium (IFM), ROS levels were significantly lower (−35% less) compared to the levels in cultures RWE-treated in iron-containing medium (FIG. 4A). Importantly, LF further decreased RWE-induced ROS levels in cells placed in IFM. Treatment of cells with Amb a 1, the most abundant allergen in RWE possessing no NAD(P)H oxidase activity, did not alter intracellular levels of ROS and LF or LF^{Fe} had no effect. DFO decreased the RWE-induced increase in cellular ROS levels by approximately 40% (FIG. 4A). O₂⁻ is dismutated to H₂O₂ by SOD and by the iron-catalyzed Haber-Weiss reaction. Accordingly, LF (and DFO) also significantly (P=0.01) inhibited H₂O₂ accumulation (FIG. 4B). To further support validity of these finding, selected experiments were carried out using NHBE cells. As summarized in FIG. 4C, LF but not LF^{Fe} significantly decreased RWE-induced ROS levels in NHBE cells.

ROS, primarily OH radicals are known to elicit, in vivo and in vitro oxidative decomposition of lipids (in the airway lining fluid, cells membrane lipids). This leads to the formation of mixture of aldehydic end-products, including MDA and 4-HNE, which we showed to be present in BAL fluid after RWE challenge of mice. To elucidate whether LF inhibits formation of OH radicals and consequently formation of lipid peroxidation products we determined 4-HNE+MDA levels in the BAL fluid from mice challenged with RWE±LF (or i DFO). Analysis of BAL fluids showed that RWE challenge increased 4-HNE+MDA levels (FIG. 4E). LF significantly inhibited this increase. DFO had similar effect. OH is also formed from H₂O₂ in the presence of iron. Results in FIG. 4D show that LF (and also DFO) partially inhibited the rapid (within 30 min) increase in H₂O₂ levels in the BAL after RWE challenge of animals. These data strongly indicate that iron-mediated dismutation of O₂⁻ and H₂O₂ into OH may serve the bases of lipid radicals formation.

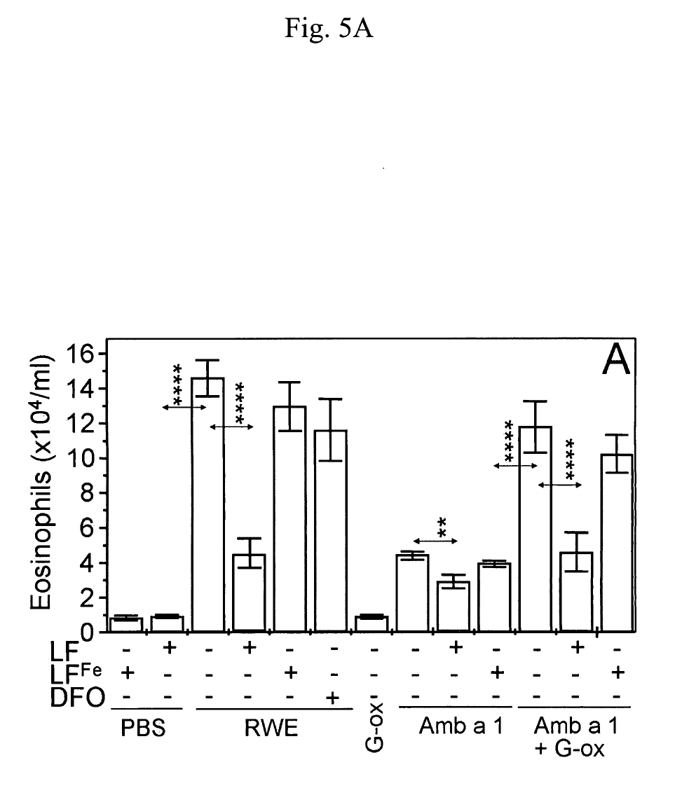

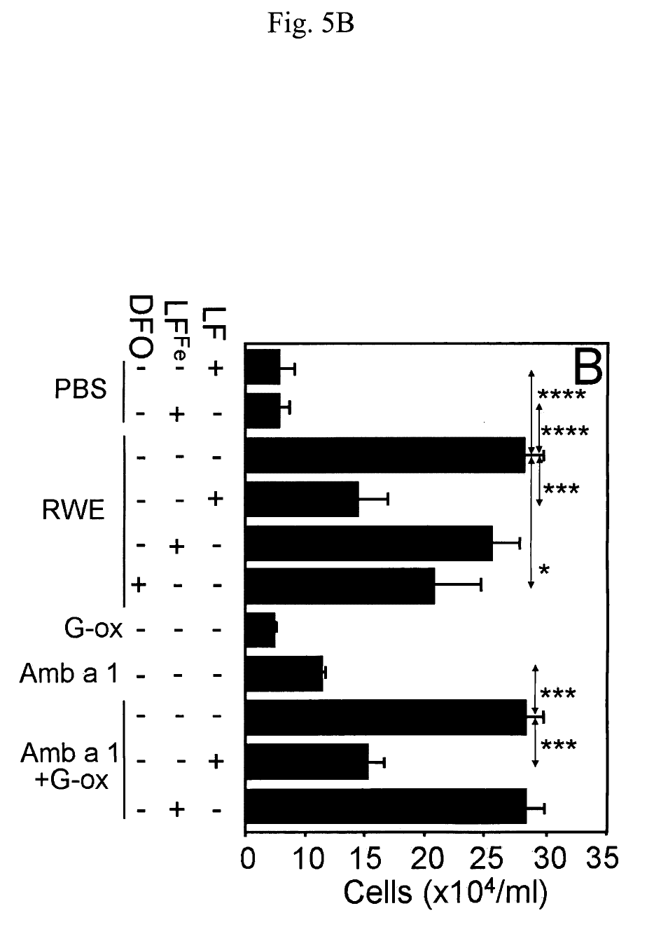

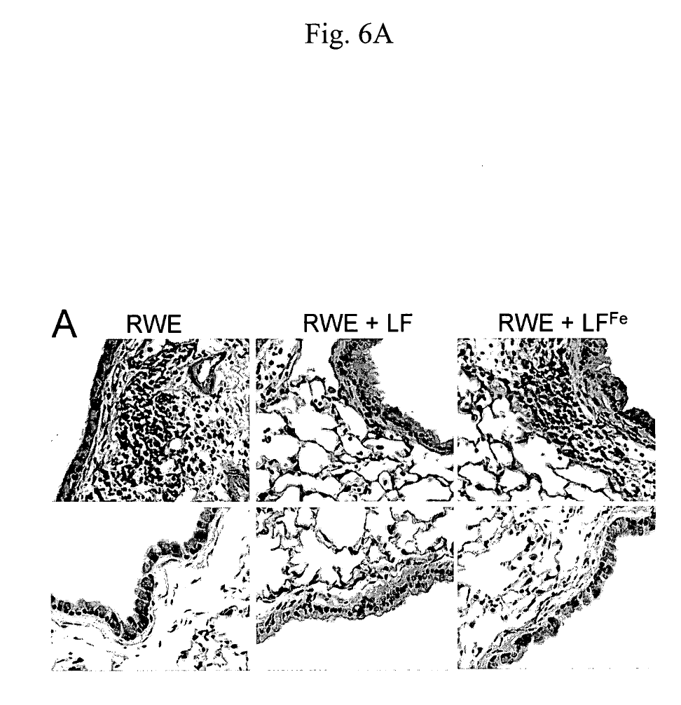

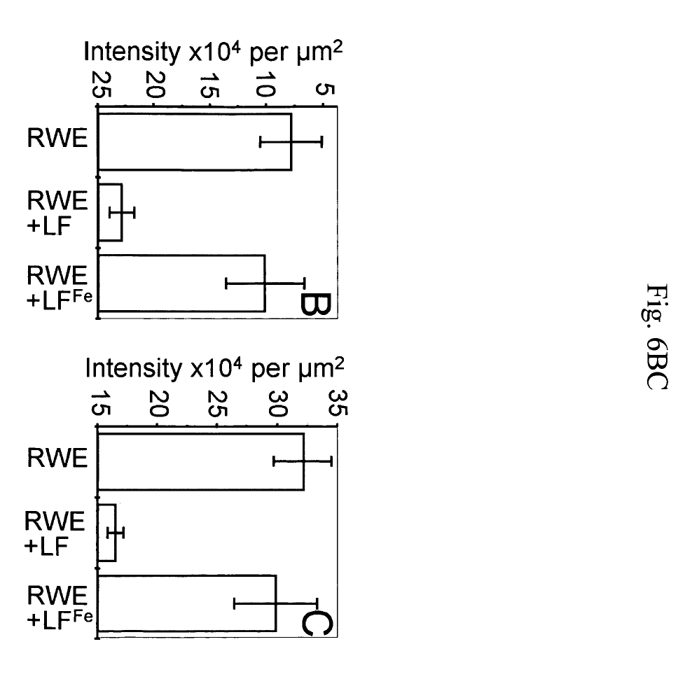

Effect of lactoferrin on RWE-induced accumulation of inflammatory cells in the airways: To explore the possibility that LF may decrease RWE-induced allergic airway inflammation, an experimental mouse model was used. When RWE-sensitized mice were challenged with RWE (100 μg per challenge) robust airway inflammation was observed as determined by accumulation inflammatory cells in the BAL compartment as well as in subepithelial locations (FIGS. 5A,B and 6). The BAL of mice prior to challenge contained primarily macrophages/monocytes (99%±0.9) and low number of eosinophils 0.1%±0.05 and neutrophils (0.1%±0.05). In a full blown inflammation; however, 47%±6.2 cells were eosinophils, 52%±3.8 macrophages/monocytes and neutrophils (1%±0.2). When RWE was administered together with LF (100 μg) there was a moderate accumulation of inflammatory cells in the BAL compartment (FIGS. 5A, B) and in the subepithelium (FIG. 6A), the latter was quantified by morphometric analyses of sections (FIG. 6B). Likewise, LF significantly decreased the RWE-induced formation of mucin-producing cells (FIGS. 6A,C). There was no statistically significant effect of LF^{Fe} (FIGS. 5A,B; FIGS. 6A,B,C). When DFO was added with RWE, there was only a slight decrease in inflammation (FIGS. 5A,B).

The oxidatively inactive Amb a 1 (25 μg) induced low-grade airway inflammation (FIGS. 5A,B). Surprisingly, LF decreased Amb a 1-induced inflammation substantially. LF^{Fe} had no effect. When Amb a 1 was administered together with the ROS-generating G-ox, eosinophil accumulation in BAL was significantly increased (FIGS. 5A,B). G-ox itself did not cause inflammatory cell accumulation in airways (FIGS. 5A,B). When G-ox plus Amb a 1 and LF were administered together, the number of inflammatory cells in BAL decreased significantly (FIGS. 5A,B). LF^{Fe} showed no statistically significant effect.

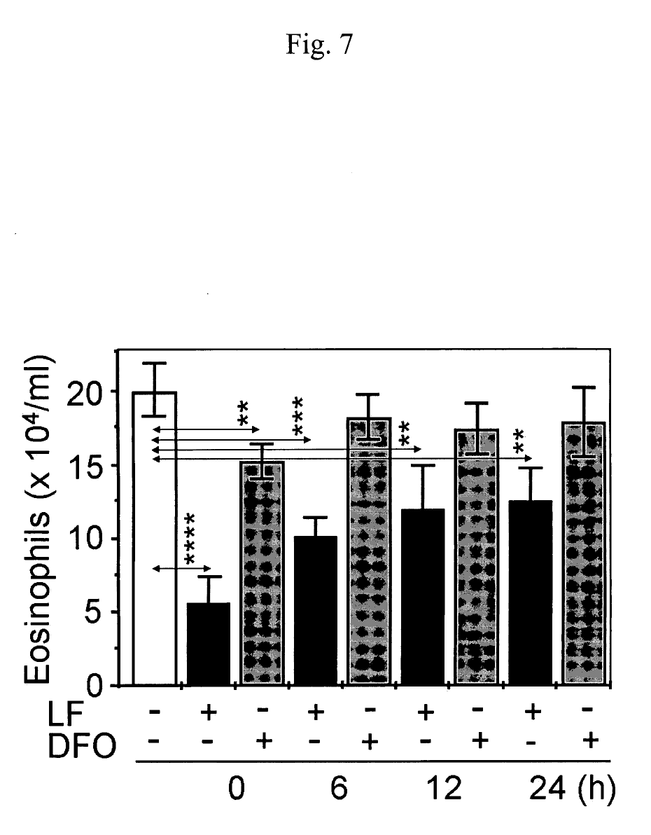

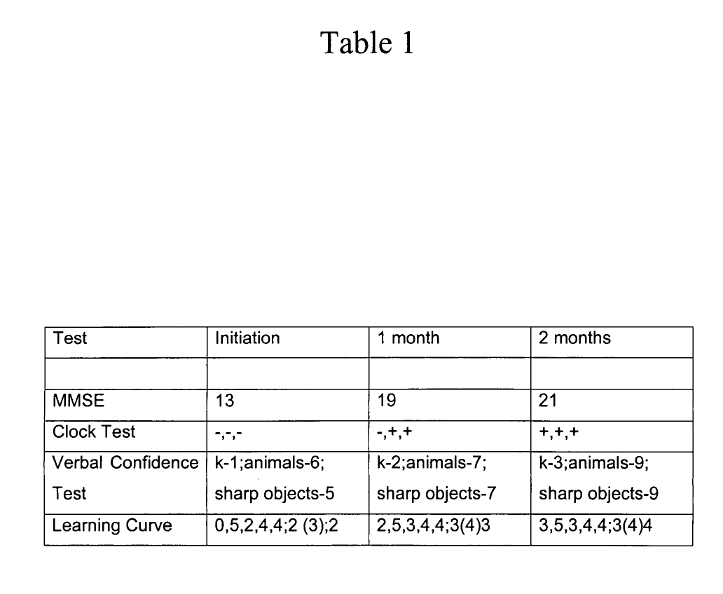

To sort out whether RWE challenge-induced immediate changes (decrease in oxidative levels) and/or some undefined inflammatory event(s) are also influenced by LF, we administered LF (or DFO) either simultaneously with RWE or 6, 12 or 24 hr following RWE-challenge. As shown in FIG. 7, LF was most effective when administered concurrently with RWE (0 hr). In addition, when LF was given 6, 12 or 24 hr after challenge also reduced inflammatory cell accumulation (FIG. 7). DFO was significantly less effective compare to LF when co-administered with RWE. Addition of DFO at later time points had no effect. These data together suggest that LF has both iron dependent and iron independent activity regarding development of allergic inflammatory processes.

In this example, we demonstrate that LF decreases allergic airway inflammation induced by RWE and Amb a 1. Specifically, we show that LF significantly decreases inflammatory cell accumulation in the airways induced by the redox-active RWE. Co-challenge of mice with RWE and the iron-binding DFO resulted in only a partial decrease in the level of inflammation. LF also decreased allergic inflammation induced by the non-redox Amb a 1. When mice were co-challenged with Amb a 1 and G-ox the level of inflammation increased by 3-fold (P=0.0001) compared to Amb a 1 alone. Remarkably, LF decreased G-α-mediated augmentation of Amb a 1-induced airway inflammation to level observed for Amb a 1 alone, while DFO had a less pronounced effect. This important observation demonstrates that LF inhibits inflammatory process through both iron dependent and iron independent mechanisms. Mucus hypersecretion by goblet cells is one of the major causes of airway obstruction in patients with allergic asthma. RWE challenge of mice caused an intense accumulation of mucin-producing cells (FIGS. 6A,C). Importantly, administration of LF with RWE significantly decreased accumulation of mucin-producing goblet cells to nearly background level in the airway epithelium shown by PAS stain.

Our cell culture studies show that LF indeed possesses an antioxidant activity, which is nearly sufficient to protect cells from RWE-mediated oxidative stress. When cells were transferred into iron-free medium RWE-induced ROS levels were lower suggesting a significant role of iron in conversion of O₂⁻ into highly-reactive species. Most importantly LF was more effective in decreasing cellular ROS levels then absence of iron in the culture medium or addition of the iron-binding DFO to the cells. Similar results were observed when cells were LF-treated and exposed to G-ox, a ROS producing enzyme. Because in iron-free medium LF further decreased RWE-induced cellular ROS levels it suggests that iron-binding is only one of the actions of LF. LF^{Fe} showed only marginal effects due to the fact that it cannot bind iron that remain available to participate as a catalyst for the generation of the OH. In our previous studies, we showed that 4-HNE and MDA levels significantly increased in airway lining fluid after RWE challenge. Indeed, partial reduction of molecular oxygen by pollen NAD(P)H oxidases yields O₂⁻, which in the presence of iron is converted to OH (the Fenton and Haber-Weiss reaction). O₂⁻ has a relative long half life, a limited reactivity with some proteins, but not with lipids or DNA. On the other hand, OH is extremely active oxidant, which is known to participate in lipid peroxidation and causes protein oxidation and DNA damage in cells. Our data show that LF significantly lowers RWE-induced increase in 4-HNE+MDA level, thus appears to inhibit conversion of O₂⁻ to highly reactive species.

In parallel experiment, we also show that LF is most effective in decreasing inflammation when added together with RWE. Interestingly, when added at later time points after RWE challenge its effect is still significant. This phenomenon indicates that in addition to binding of iron, LF may have other actions for decreasing allergic immune responses, including reduction of the expression of pro-inflammatory mediators or binding various metabolically important molecules.

Research into free iron metabolism in the lower respiratory tract shows' that 80% of iron is present in the airway lining cells, especially in macrophages, and 20% in the epithelial lining fluid of the lung. LF is also present in airway lining fluid, however, its concentration is approximately 10-times lower than that of other iron chelators e.g., transferritin. Because LF supplementation along with allergen challenge significantly decreases RWE-induced oxidative stress levels (e.g., 4-HNE and MDA) in the BAL and RWE-induced airway inflammation it suggests that LF along with its other properties may have a particular role in iron metabolism in airways different from other iron-binding proteins. It appears that the free iron exacerbates RWEs' NAD(P)H oxidase generated oxidative stress. Redox active pollens or subpollen particles, and exogenous oxidant substances (ozone, cigarette smoke, NO₂), oxidant particulate matters (found in diesel, cigarette smoke) exacerbate antigen-induced airway inflammations. Therefore, our data suggest that LF can ameliorate impact of other oxidants on antigen-induced inflammation and differentiate lactoferrin from simple iron sequestering molecule. Again, this novel observation holds a promise for the therapeutic utility of LF in human allergic inflammatory disorders.

### Treatment of Autoimmune Disorders

According to the present invention, exogenous lactoferrin is used to modulate the molecular events during development of autoimmune disorders in humans. In a preferred embodiment of the present invention, lactoferrin is used for treatment of multiple sclerosis. MS is the autoimmune disorder. There is growing evidence suggesting that autoimmune T cell responses to myelin basic protein (MBP) are engaged in the pathogenesis of MS. MS is characterized by disseminated patches of demyelination in the brain and spinal cord, resulting in multiple and varied neurologic symptoms. The myelin sheath, a lipid-rich membrane, both insulates and enhances conduction in nerve axons. Nerves can only conduct pulses of energy efficiently if covered by myelin.

This process of demyelination usually starts in adolescence, but the first symptoms may not be experienced until the early to mid-twenties—this is when the diagnosis is usually made. So the affected person is asymptomatic for years, in spite of the development of lesions, because nerve conduction can still occur in spite of large areas of demyelination. Studies with NMR (Nuclear Magnetic Resonance) have permitted researchers to observe the appearance of lesions days before the appearance of symptoms during a period of exacerbation, and the disappearance of these fresh plaques during the period of remission that follows. The exact mechanism(s) of demyelination in multiple sclerosis is still unresolved, both antigen-specific and—non-specific events having the potential to generate the myelinolytic process.

The effectiveness of lactoferrin in the treatment of multiple sclerosis is illustrated in Example 7 and 8.

### EXAMPLE 7—MS-Large Population Clinical Studies

In our placebo controlled clinical trial, LF was administered to patients orally, twice daily (25 mg/dose), for seven consecutive days. Six of the patients suffer from MS and 24 were diagnosed with persistent fatigue. Blood samples were taken on 1 day before treatment, 1 day, and 7 days after cessation of the treatment. The leukocytes were isolated from the whole blood, the cultures were established and cells stimulated with phytoheamoglutinin (PHA) and lipopolysaccharide (LPS) overnight. In the plasma the following parameters were measured: endogenous lactoferrin, NO and cortisol. In the unstimulated and stimulated cell cultures the activities of IFN gamma, TNF alpha, IL-6, and IL-10 were determined. In addition, the blood smears were stained and the percentage of main cell types was determined.

The production of IL-10 was increased in MS patients treated with lactoferrin by 8.13× on average (individual increases: 10×; 32×; 4×; 17×; 7×). On the other hand in the placebo group, IL-10 activity dropped by 34%. The dramatic increase in the IL-10 production, was associated with changes in IFN gamma production, which dropped on average by 4× in MS patients treated with lactoferrin (from 186 pg/ml to 46 pg/ml). The stimulation was observed in only one MS patient. In the placebo group the changes in the production of IFN gamma were minor. Elevation of serum cortisol would be advantageous in diminishing manifestations of MS. In fact, our clinical studies showed that cortisol has been increased in all MS patients treated with lactoferrin. In placebo group, the level of cortisol dropped by 14%. More important the changes in the immunological parameters were correlated with improvement of overall wellness and complete release from common fatigue.

### EXAMPLE 8—MS Individual Treatment

Lactoferrin tablets (Example 2) were administered twice daily for 12 months to an adult woman (42 years old) with a history of disseminated sclerosis (subject A). The patient was evaluated three times: at the initiation, 6 months into the therapy and 11 months after initiation of the treatment, by using NMR imaging analysis. At the initiation of therapy, subject A experienced difficulties with walking and performing routine daily exercises. NMR analysis showed significant demyelination by number of hyper intensive centers in both brain and spinal cord. Six months into the therapy subject A was able to walk and perform most of daily duties. The NMR showed less hyper intensive centers in brain. After the treatment, subject A reported no limitation on daily duties and exercises and the NMR confirmed less lesions in brain and spinal cord. The rate of demeylination was significantly reduced in subject A after one year lactoferrin treatment.

### EXAMPLE 9—RT Treatment

Lactoferrin tablets (Example 2) were self-administered by subject B, an adult woman with a long history of rheumatoid arthritis. Tenderness in all active joints and deformities in fingers, wrists and elbows were very visible signs of inflammation. Over several years subject B had experienced no relief from medications prescribed by physicians. Pain relief was observed as soon as a regime was initiated in which two tablets of lactoferrin were taken orally each day. Over three months the morning stiffness of joints improved to the point at which symptoms were absent. Also, joints deformities, especially those on fingers, were significantly reduced.

### EXAMPLE 10—CFS Treatment

Lactoferrin tablets (Example 2) were self-administered by subject C, an adult male with a history of persistent fatigue. In general, subject C reported fluctuating level of energy from time to time. Also, tiredness and muscle weakness renders subject C incapable of normal activities of daily living. Over several months subject C had experienced no relief from over the counter medications. After six day treatment with 2 tablets a day, subject C reported increased level of energy and no muscle weakness. Within 2 weeks into treatment subject C declared free of any symptoms previously described as fatigue.

These data demonstrate that lactoferrin given orally in the range of 25-150 mg daily, is an effective and safe treatment to alleviate the symptoms of autoimmune disorders, in particular multiple sclerosis, rheumatoid arthritis and CFS in humans.

### Treatment of Neurodegenerative Disorders

According to the present invention, exogenous lactoferrin is used to modulate the molecular events during development of neurodegenerative disorders in humans. In another preferred embodiment of the present invention, lactoferrin is used for treatment of Alzheimer's disease. AD is slowly progressive neurodegenerative disorder, with a mean survival interval of 9 to 10 years following onset. The first symptoms of AD often include memory loss, temporal and geographical disorientation, and language deficits. As the disease progresses, these deficits become more severe and personality changes are common, including withdrawal from social settings and impairments in judgment and problem solving. Sensory, motor, and primary visual functions are typically not lost until the final stages of the disease. The two pathognomonic lesions of Alzheimer's disease are senile plaques (SPs) and neurofibrillary tangles (NFTs). In addition to SPs and NFTs, the most prominent feature of AD pathology is massive neuronal loss, primarily in the hippocampus. Neurofibrillary tangles are intraneuronal lesions composed primarily of the microtubule-associated protein tau. The major constituents of senile plaques are amyloid fibrils made of 3943 amino acid amyloid-β (Aβ peptides. There are two types of senile plaques: neuritic plaques, which are surrounded by dystrophic neurites and diffuse plaques, which are not accompanied by abnormal neurites. The neuritic and diffuse plaques may contain different populations of Aβ peptides. The neuritic plaques contain mostly A β42, whereas diffuse plaques are made of A β40. Although very little is known about the mechanisms by which these different types of senile plaques are generated, the presence of A β40 and A β42 in the CSF of normal and AD patients suggests that Aβ is constitutively produced and secreted in vivo.

The effectiveness of lactoferrin in the treatment of the neurodegenerative disorders is illustrated in the following Examples:

### EXAMPLE 11—AD Treatment

Lactoferrin tablets (Example 2) were administered twice daily for 3 months to a 61 year old male with a history of increasing memory problems and lack of focus (subject D). The patient was diagnosed with a moderate Alzheimer's disease. The effectiveness of lactoferrin treatment was evaluated two times following the initial diagnose: 1 month into the therapy and 2 months after initiation of the treatment, by using standard psychological tests, including Mini Mental State Examination (MMSE). A transient occurrence of excitement was reported by subject D during first week of treatment. An improvement in memorizing daily activities was reported after two weeks of treatment, followed by further revitalization as shown in table 1.

A continuous regression (improvement) in dementia has been reported by subject D for one year now.

### EXAMPLE 12—Stroke/TIA Treatment

Lactoferrin tablets (Example 2) were self-administered by subject E, an adult woman suffering from the transient ischemic attack (TIA). Lactoferrin tablets were administered orally immediately after experiencing numbness in right hand, difficulties to walk and slurred speech. Following administration of first tablet, subject E reported immediate occurrence of excitement in the experience of relief from the numbness. Further improvement in walking and articulate speech was noticed within 15 minutes following an initial attack. Subject E continued self-administration of lactoferrin tablets twice daily for 1 month and did not report reoccurrence of TIA or stroke for 3 years.

These data demonstrate that lactoferrin given orally in the range of 25-150 mg daily, is an effective and safe treatment to alievate the symptoms of neurodegenerative disorders, in particular AD and stroke in humans.

It is understood that the examples and embodiments described herein are for illustrative purposes only and that various modifications or changes in light thereof will be suggested to persons skilled in the art and are to be included within the spirit and purview of this application and scope of the appended claims.

What we claimed is:

1. A method for alleviation of immune dissonance arising from oxidative stress in a subject having or at risk of developing said immune dissonance conditions, said method comprising administering an effective amount of isolated and purified lactoferrin, wherein said lactoferrin comprises iron-free or partially iron-saturated lactoferrin in a pharmaceutically or nutritionally acceptable carrier.

2. A method according to claim 1, wherein said isolated and purified lactoferrin is bovine lactoferrin.

3. A method according to claim 1, wherein said isolated and purified lactoferrin is human lactoferrin.

4. A method for alleviation of dementia in a subject having or at risk of developing said dementia conditions, said method comprising administering an effective amount of isolated and purified lactoferrin, wherein said lactoferrin comprises iron-free or partially iron-saturated lactoferrin in a pharmaceutically or nutritionally acceptable carrier.

5. A method according to claim 4, wherein said isolated and purified lactoferrin is bovine lactoferrin.

6. A method according to claim 4, wherein said isolated and purified lactoferrin is human lactoferrin.

7. A method for alleviation of demyelination in a subject having or at risk of developing said demyelination conditions, said method comprising administering an effective amount of isolated and purified lactoferrin, wherein said lactoferrin comprises iron-free or partially iron-saturated lactoferrin in a pharmaceutically or nutritionally acceptable carrier.

8. A method according to claim 7, wherein said isolated and purified lactoferrin is bovine lactoferrin.

9. A method according to claim 7, wherein said isolated and purified lactoferrin is human lactoferrin.

10. A method for alleviation of inflammatory cells accumulation due to allergic reaction in a subject at risk or developing said allergic reaction conditions, said method comprising administering an effective amount of isolated and purified lactoferrin, wherein said lactoferrin comprises iron-free or partially iron-saturated lactoferrin in a pharmaceutically or nutritionally acceptable carrier.

11. A method according to claim 10, wherein said isolated and purified lactoferrin is bovine lactoferrin.

12. A method according to claim 10, wherein said isolated and purified lactoferrin is human lactoferrin.

13. A method for lessening of allergen-induced bronchial airway obstruction in a subject at risk or developing said airway obstruction, said method comprising administering an effective amount of isolated and purified lactoferrin, wherein said lactoferrin comprises iron-free or partially iron-saturated lactoferrin in a pharmaceutically or nutritionally acceptable carrier.

14. A method according to claim 13, wherein said isolated and purified lactoferrin is bovine lactoferrin.

15. A method according to claim 13, wherein said isolated and purified lactoferrin is human lactoferrin.

16. A method for restoring and maintaining central nervous system health in a subject at risk or developing an oxidative stress induced central nervous system impairment, said method comprising administering an effective amount of isolated and purified lactoferrin, wherein said lactoferrin comprises iron-free or partially iron-saturated lactoferrin in a pharmaceutically or nutritionally acceptable carrier.

17. A method according to claim 16, wherein said isolated and purified lactoferrin is bovine lactoferrin.

18. A method according to claim 16, wherein said isolated and purified lactoferrin is human lactoferrin.
